(19) United States
(12) Patent Application Publication
(10) Pub. No.: US 2026/0016413 A1
(43) Pub. Date: Jan. 15, 2026

(54) PHYSICAL QUANTITY MEASUREMENT APPARATUS

(71) Applicant: Asahi Kasei Microdevices Corporation, Chiyoda-ku, Tokyo (JP)

(72) Inventor: Yuji GODA, Chiyoda-ku, Tokyo (JP)

(73) Assignee: Asahi Kasei Microdevices Corporation, Chiyoda-ku, Tokyo (JP)

(21) Appl. No.: 19/099,047

(22) PCT Filed: Aug. 1, 2023
(86) PCT No.: PCT/JP2023/028160

§ 371 Date: Jan. 28, 2025

(30) Foreign Application Priority Data
Aug. 2, 2022 (JP) 2022-123384

(51) Int. Cl. G01N 21/59; G01N 33/00

(52) U.S. Cl. CPC G01N 21/5907; G01N 33/0027

(57) ABSTRACT

A physical quantity measurement apparatus capable of measuring with high accuracy is provided. A physical quantity measurement apparatus (1) includes a first substrate (10) and a second substrate (20) and measures a physical quantity of an object to be measured. The first substrate includes a sensing part (13) on a front surface (10a) and a surface electrode on a back surface, the sensing part being configured to output a signal according to the physical quantity. The second substrate includes a side electrode. When the first substrate is viewed from a direction perpendicular to the front surface or the back surface of the first substrate, at least a portion of the first substrate overlaps the second substrate. The surface electrode is electrically connected to the side electrode.

### TECHNICAL FIELD

The present disclosure relates to a physical quantity measurement apparatus.

### BACKGROUND

A physical quantity measurement apparatus that measures a physical quantity of an object to be measured may be configured by a sensing part (sensor) and a main controller installed on different substrates, with the main controller controlling the entire apparatus, including the operation of the sensing part. Demand exists for further miniaturization of the physical quantity measurement apparatuses, but when pin headers, for example, are used for electrical connection between substrates, further miniaturization is difficult because of the space created by insulating portions. For example, Patent Literature (PTL) 1 discloses a printed circuit board with side terminals that can be soldered without use of pin headers.

### CITATION LIST

### Patent Literature

PTL 1: JP H9-18104 A

### SUMMARY

### Technical Problem

Here, in the technology of PTL 1, the module board and the mother board have side terminals to be connected to lands of the other board. Therefore, when the technology of PTL 1 is applied to a physical quantity measurement apparatus, the side of the module board on which the sensing part is mounted is also soldered. If soldering is performed near the sensing part, the sensing part or the area near the sensing part is exposed to high temperatures. This changes the characteristics of the sensing part due to thermal and mechanical effects, and the measurement accuracy of the physical quantity measurement apparatus degrades. If soldering is performed near the sensing part, vaporized flux may also adhere to the sensing part and change the characteristics of the sensing part, resulting in a decrease in the measurement accuracy of the physical quantity measurement apparatus.

In light of these facts, it is an aim of the present disclosure to provide a physical quantity measurement apparatus capable of measuring with high accuracy.

### Solution to Problem

(1) A physical quantity measurement apparatus according to an embodiment is
a physical quantity measurement apparatus that includes a first substrate and a second substrate and measures a physical quantity of an object to be measured, wherein
the first substrate includes a sensing part on a front surface and a surface electrode on a back surface, the sensing part being configured to output a signal according to the physical quantity,
the second substrate includes a side electrode,
when the first substrate is viewed from a direction perpendicular to the front surface or the back surface of the first substrate, at least a portion of the first substrate overlaps the second substrate, and
the surface electrode is electrically connected to the side electrode.

(2) As an embodiment of the present disclosure, in (1),
the side electrode is a castellated hole.

(3) As an embodiment of the present disclosure, in (1) or (2),
the surface electrode includes a through-hole electrode.

(4) As an embodiment of the present disclosure, in any one of (1) to (3),
the sensing part includes a light emitting element and a detection element that detects an amount of absorption, by the object to be measured, of light emitted from the light emitting element, and the physical quantity is measured on the basis of the amount of absorption.

(5) As an embodiment of the present disclosure, in (4),
the light is infrared light.

(6) As an embodiment of the present disclosure, in any one of (1) to (5),
the physical quantity is a concentration of a gas to be detected.

(7) As an embodiment of the present disclosure, in any one of (1) to (6),
at least one of the first substrate and the second substrate includes at least one of a guide and a mark used to align the first substrate and the second substrate.

(8) As an embodiment of the present disclosure, in any one of (1) to (7),
the first substrate includes the sensing part and a component on the front surface, and
at least a portion of the sensing part and the component is provided to be in overlap with the side electrode when the first substrate is viewed from a direction perpendicular to the front surface or the back surface of the first substrate.

### Advantageous Effect

According to the present disclosure, a physical quantity measurement apparatus capable of measuring with high accuracy can be provided.

### BRIEF DESCRIPTION OF THE DRAWINGS

In the accompanying drawings:

FIG. 1 is a perspective diagram illustrating an example configuration of a physical quantity measurement apparatus according to an embodiment of the present disclosure;

FIG. 2 is a perspective view of the physical quantity measurement apparatus in FIG. 1 viewed from the back side;

FIG. 3 is a plan view illustrating the upper surface of the physical quantity measurement apparatus in FIG. 1;

FIG. 4 is a plan view of the front surface of a first substrate;

FIG. 5 is a plan view of the back surface of the first substrate in FIG. 4;

FIG. 6 is a cross-sectional view of the first substrate illustrated in FIG. 4, including a light guiding member;

FIG. 7 is a diagram illustrating an example of a surface electrode;

FIG. 8 is a diagram illustrating an example of a surface electrode;

FIG. 9 is a diagram illustrating an example of a surface electrode;

FIG. 10 is a diagram illustrating an example of a surface electrode;

FIG. 11 is a plan view illustrating the front surface of a second substrate;

FIG. 12 is a plan view of the back surface of the second substrate in FIG. 11;

FIG. 13 is a diagram illustrating an example of a side electrode;

FIG. 14 is a diagram illustrating an example of a side electrode;

FIG. 15 is a diagram illustrating an example of a side electrode; and

FIG. 16 is a diagram illustrating an example of a side electrode.

### DETAILED DESCRIPTION

A physical quantity measurement apparatus according to an embodiment of the present disclosure is described below with reference to the drawings. Identical or equivalent portions in the drawings are labeled with the same reference signs. In the explanation of the embodiments, a description of identical or equivalent portions is omitted or simplified as appropriate. These drawings are schematic. For example, the thickness, length, and the like differ from the actual dimensions. The technical concept of the present disclosure can be modified in various ways within the technical scope defined by the claims. The following embodiments are not intended to limit the contents of the present disclosure. Furthermore, not all combinations of features described in the embodiments are necessarily essential to the solution to the problem.

FIG. 1 is a perspective diagram illustrating an example configuration of a physical quantity measurement apparatus 1 according to the present embodiment. In FIGS. 1 to 4, the below-described light guiding member 15 is excluded (made transparent) to illustrate the arrangement of the light emitting element 11, the detection element 12, and the like. The physical quantity measurement apparatus 1 includes a first substrate 10 and a second substrate 20 and measures a physical quantity of an object to be measured. The first substrate 10 and the second substrate 20 are substrates for mounting components and are electrically connected to configure the physical quantity measurement apparatus 1. In the present embodiment, the first substrate 10 and the second substrate 20 are described as being printed circuit boards made of hard resin. The first substrate 10 and the second substrate 20 are not limited in type, however, and may be different types. In the present embodiment, the physical quantity measurement apparatus 1 is described as a gas sensor that takes the concentration of a gas to be detected as the physical quantity. However, the physical quantity is not limited to a specific physical quantity.

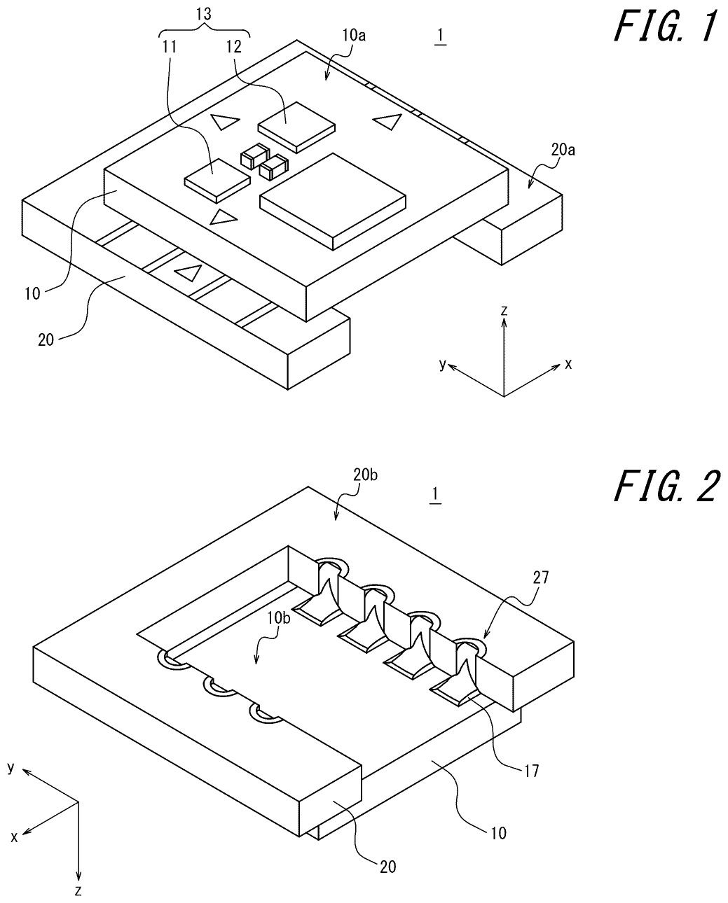

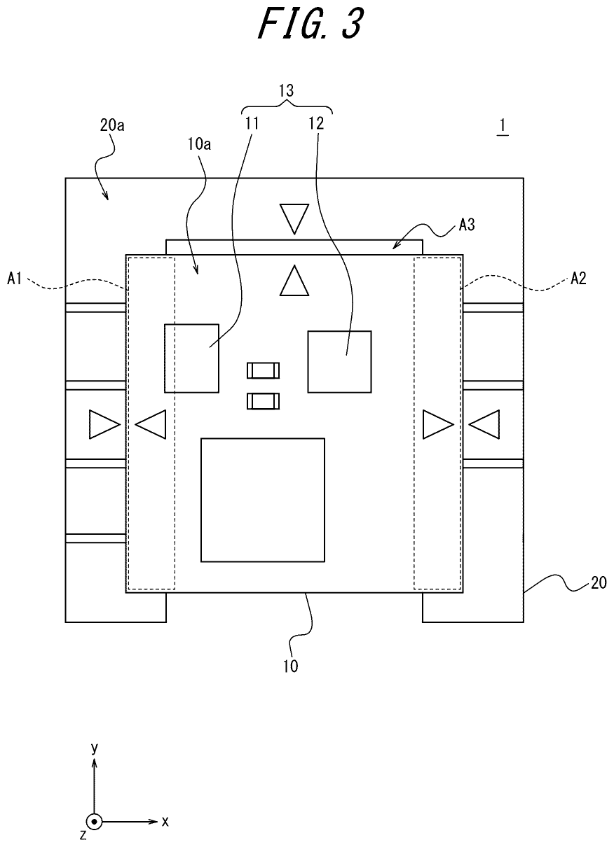

In the following description, the front surface 10a of the first substrate 10 is one of the surfaces (main surfaces) with the largest area of the first substrate 10 and is the surface that is farther from the second substrate 20 when the physical quantity measurement apparatus 1 is configured. The back surface 10b of the first substrate 10 (see FIG. 2) is a different main surface of the first substrate 10 than the front surface 10a. The front surface 20a of the second substrate 20 is one of the surfaces (main surfaces) with the largest area of the second substrate 20 and is the surface closer to the first substrate 10 when the physical quantity measurement apparatus 1 is configured. The back surface 20b of the second substrate 20 (see FIG. 2) is a different main surface of the second substrate 20 than the front surface 20a. FIG. 1 is a perspective diagram of the physical quantity measurement apparatus 1 viewed from the side of the front surface 10a and the front surface 20a. FIG. 2 is a perspective diagram of the physical quantity measurement apparatus 1 in FIG. 1, viewed from the side of the back surface 10b and the back surface 20b. FIG. 3 is a plan view illustrating the upper surface of the physical quantity measurement apparatus 1, viewed from the side of the front surface 10a and the front surface 20a.

In FIGS. 1 to 3, right-handed Cartesian coordinates corresponding to the orientation of the physical quantity measurement apparatus 1 are set. The z-axis direction is the height direction of the physical quantity measurement apparatus 1 and is perpendicular to the front surface 10a or back surface 10b of the first substrate 10. The z-axis direction is also the stacking direction of the first substrate 10 and second substrate 20. The first substrate 10 is on the positive side along the z-axis relative to the second substrate 20. The y-axis direction corresponds to the vertical direction of the main surface of the first substrate 10, which is rectangular. The x-axis direction corresponds to the transverse direction (width direction) of the main surface of the first substrate 10. The xy-plane is parallel to the main surface of the first substrate 10. These Cartesian coordinates are used in common in the other drawings referred to below, and the axes of these Cartesian coordinates may be used to describe positional relationships.

As described above, the physical quantity measurement apparatus 1 is configured to include a first substrate 10 and a second substrate 20 that are electrically connected. The first substrate 10 has a sensing part 13 that outputs a signal according to a physical quantity on the front surface 10a. In the present embodiment, the sensing part 13 includes a light emitting element 11, a detection element 12, and the like. The sensing part 13 may output analog signals such as current or voltage in accordance with the physical quantity to be measured and may output values, yielded by converting those analog signals to physical quantities, as digital signals or as other analog signals. Details of the light emitting element 11 and the detection element 12 are provided below. The first substrate 10 has a surface electrode 17 on the back surface 10b, as illustrated in FIG. 2. The second substrate 20 has a side electrode 27 that is between the front surface 20a and the back surface 20b, and not on the outer periphery of the second substrate 20 but on a part of the side surface where the notch is provided, as illustrated in FIG. 2. Here, the notch may be a notch provided to connect with the periphery of the second substrate, as illustrated in FIG. 2, or may be an internal notch. The size of the notch should be such that one side is at least as large as the thickness of the substrate in plan view. One side is preferably at least five times the size of the substrate, as this allows a plurality of side electrodes to be provided in a single notch. The surface electrode 17 is electrically connected to the side electrode 27 by soldering. Here, one or more surface electrodes 17 and one or more side electrodes 27 are provided. In the present embodiment, a plurality of surface electrodes 17 and a plurality of side electrodes 27 that are the same in number as the surface electrodes 17 are provided, but the number of surface electrodes 17 and the number of side electrodes 27 may be different. Not all of the surface electrodes 17 need to be soldered to the side electrodes 27. For example, there may be one or more surface electrodes 17 that are not electrically connected to the side electrodes 27. Also, not all of the side electrodes 27 need to be soldered to the surface electrodes 17. For example, there may be one or more side electrodes 27 that are not electrically connected to the surface electrodes 17.

The physical quantity measurement apparatus 1 has a configuration such that when the first substrate 10 is viewed from the z-axis direction, at least a portion of the first substrate 10 overlaps the second substrate 20. In the present embodiment, the physical quantity measurement apparatus 1 has an area A1 and an area A2 where the first substrate 10 and the second substrate 20 overlap, as illustrated in FIG. 3. Here, the portion where the surface electrode 17 and the side electrode 27 are electrically connected is located in the area where the first substrate 10 and the second substrate 20 overlap. The number of areas where the first substrate 10 and the second substrate 20 overlap is not limited to two. For example, the area of overlap may be only the area A1, or only the area A2. In a case in which the first substrate 10 and the second substrate 20 overlap in a plurality of areas, not all of the areas need to be electrically connected via the surface electrode 17 and the side electrode 27. The first substrate 10 and the second substrate 20 may be physically connected by adhesive or the like. The area where the first substrate 10 and the second substrate 20 overlap may be both electrically connected via the surface electrode 17 and the side electrode 27 and physically connected by adhesive or the like. The physical quantity measurement apparatus 1 may be configured to have a portion (gap) in which, when the first substrate 10 is viewed from the z-axis direction, at least a portion of the first substrate 10 does not overlap the second substrate 20. In the present embodiment, the physical quantity measurement apparatus 1 has an area A3, which is a gap, as illustrated in FIG. 3. Here, the area where the first substrate 10 and the second substrate 20 do not overlap corresponds to a portion where no surface electrode 17 or side electrode 27 exists, or where the surface electrode 17 and the side electrode 27 are not electrically connected. The number of areas where the first substrate 10 and the second substrate 20 do not overlap is not limited to one. As described below in detail, this configuration of the physical quantity measurement apparatus 1 according to the present embodiment enables measurement with high accuracy by avoiding exposure of the sensing part 13 or the area near the sensing part 13 to high temperatures and avoiding adhesion of vaporized flux to the sensing part.

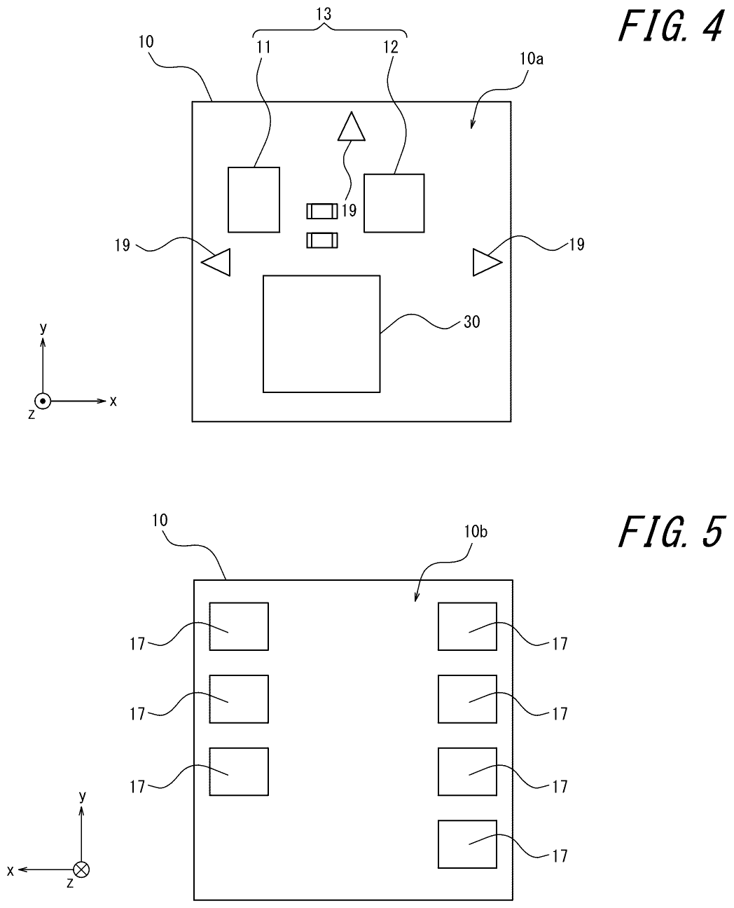

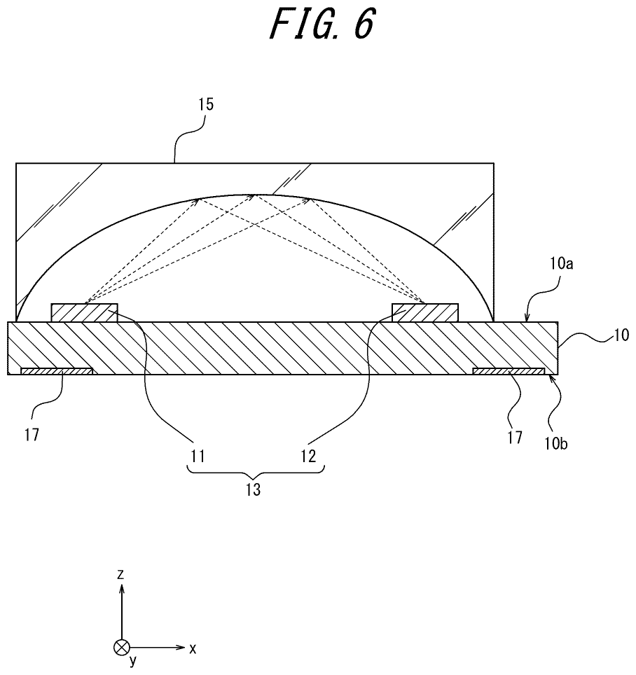

Details of the components of the physical quantity measurement apparatus 1 are described below with reference to the drawings. FIG. 4 is a plan view illustrating the front surface 10a of the first substrate 10. FIG. 5 is a plan view illustrating the back surface 10b of the first substrate 10. FIG. 6 is a cross-sectional view of the first substrate 10, including a light guiding member 15. FIG. 6 illustrates a cross-section of the first substrate 10 at the positions of the light emitting element 11 and the detection element 12.

In the present embodiment, the physical quantity measurement apparatus 1 is configured by the sensing part 13 for detecting the concentration of a gas to be detected and a main controller that controls the entire apparatus including the operation of the sensing part 13. The first substrate 10 has the sensing part 13 and components such as an IC 30 (components of the gas sensor other than the sensing part 13) on the front surface 10a. In the present embodiment, the sensing part 13 includes the light emitting element 11, the detection element 12, and the light guiding member 15. The main controller is provided on the second substrate 20. As described above, the first substrate 10 and the second substrate 20 are electrically connected by soldering to configure the physical quantity measurement apparatus 1. First, the sensing part 13 and the gas sensor are described.

In the present embodiment, the physical quantity measurement apparatus 1 includes an NDIR (Non Dispersive InfraRed) type gas sensor. The NDIR type gas sensor measures the gas concentration using the detection element 12, which receives infrared light in an absorption wavelength band corresponding to the gas to be detected, and the light emitting element 11, which emits infrared light in that absorption wavelength band. In the present embodiment, the detection element 12 is a light receiving element. The physical quantity measurement apparatus 1 also includes the light guiding member 15, which guides the light emitted from the light emitting element 11 to the detection element 12, as illustrated in FIG. 6. The physical quantity measurement apparatus 1 may, for example, further include an optical filter having a wavelength selection function at a location such as the emission surface of the light emitting element 11. The optical filter may, for example, be a bandpass filter that transmits light in the absorption wavelength band of the gas to be detected. As described above, the sensing part 13 is configured to include the light emitting element 11, the detection element 12, and the light guiding member 15. The sensing part 13 may further include an optical filter.

The physical quantity measurement apparatus 1 also includes an IC 30, which is an integrated circuit (IC) that calculates the concentration of the gas to be detected, as illustrated in FIG. 4. The physical quantity measurement apparatus 1 may further include a memory, as another component, that stores data, programs, and the like used by the IC 30. The IC 30 may be omitted, and the main controller provided on the second substrate 20 may instead calculate the concentration on the basis of the signals outputted from the detection element 12. In the present embodiment, the gas sensor is configured by the sensing part 13 and components mounted on the front surface 10a of the first substrate 10.

The light emitting element 11 is a light source that emits light used to detect the gas to be detected. The light emitting element 11 is not limited as long as it outputs light that includes wavelengths absorbed by the gas to be detected. In the present embodiment, the light emitted by the light emitting element 11 is infrared light, but this configuration is not limiting. The light emitting element 11 is an LED (Light Emitting Diode) in the present embodiment but may be a semiconductor laser, a MEMS (Micro Electro Mechanical Systems) heater, or the like as other examples. The wavelength of infrared light may be between 2 μm and 12 μm. The region of 2 μm to 12 μm is a particularly suitable wavelength range for use in gas sensors, as many absorption bands specific to various gases exist in this region. For example, absorption bands exist for methane at a wavelength of 3.3 μm, carbon dioxide at a wavelength of 4.3 μm, and alcohol (ethanol) at a wavelength of 9.5 μm.

The detection element 12 is a light receiving element in the present embodiment and receives light emitted from the light emitting element 11. The detection element 12 is not particularly limited as long as it is sensitive to a band of light that includes wavelengths absorbed by the gas to be detected. In the present embodiment, the light received by the detection element 12 is infrared light, but this configuration is not limiting. The detection element 12 outputs an electric signal based on the intensity or amount of the received light, by photoelectric conversion or the like. In other words, the electric signal is a signal based on a physical quantity, which in the present embodiment is a signal based on the concentration of the gas to be detected. The electric signal is outputted to the IC 30, for example. After receiving the electric signal, the IC 30 calculates the concentration of the gas to be detected on the basis of the amount of light absorbed by the gas to be detected, which is the object to be measured. The measurement result of the gas sensor (calculated concentration of the gas to be detected) may be outputted to the main controller provided on the second substrate 20.

The light guiding member 15 is a member that guides light emitted from the light emitting element 11 to the detection element 12. The light guiding member 15 is the optical system of the gas sensor. The light guiding member 15 includes an optical member and configures an optical path from the light emitting element 11 to the detection element 12. Here, the optical member is, for example, a mirror, lens, or the like. In the present embodiment, the light guiding member 15 is provided on the front surface 10a of the first substrate 10 and forms an interior space into which gas is introduced. The light emitted from the light emitting element 11 passes through the gas in the space via the light guiding member 15 and is received by the detection element 12. If the gas in the space contains the gas to be detected, then light of a specific wavelength is absorbed according to the concentration of the gas to be detected. The concentration can thus be measured by detecting the amount of absorption.

Here, in general, the sensing part 13 is easily affected by heat. Therefore, it is not advisable to perform soldering by placing a high temperature body, such as a small tip of a soldering iron or a nozzle for selective soldering, close to the sensing part. For example, the light guide member 15 as an industrial component is often made using resin as the base material for mass production at low cost. If a high temperature body is placed close to a light guide member 15 made of resin, however, the light guide member 15 will be deformed, and the light path that should be configured by the light guide member 15 will be deformed. In addition, if the high temperature body is brought close to the light emitting element 11 and the detection element 12, which configure the sensing part 13, then the light emitting element 11 and the detection element 12 may be scorched, and their characteristics may change. If a high temperature body is brought close to the sensing part 13, generating a temperature gradient in the vicinity of the light emitting element 11, the detection element 12, and the light guide member 15, then mechanical stress is applied to the light emitting element 11, the detection element 12, and the light guide member 15 due to the difference in linear expansion coefficient of the constituent materials. The mechanical stress causes changes in the characteristics of the light emitting element 11 or detection element 12, or deformation of the light path that should be configured by the light guide member 15. Alternatively, the mechanical stress may destroy the electrical connection between the light emitting element 11 or the detection element 12 and the first substrate 10. Also, if soldering is performed near the sensing part 13, the vaporized flux ends up adhering to the surfaces of the light emitting element 11, the detection element 12, and the light guide member 15, which configure the sensing part 13. The characteristics of the sensing part 13 consequently change due to flux adhering and changing the optical characteristics, or due to the formation of a new current leakage path, which changes the electrical characteristics. In the present embodiment, the first substrate 10 has the surface electrode 17 on the back surface 10b, as illustrated in FIG. 5. The surface electrode 17 is electrically connected to the electronic components provided on the front surface 10a by wiring inside the first substrate 10 and is soldered to the side electrode 27 of the second substrate 20. The surface electrode 17 is provided only on the back surface 10b, away from the front surface 10a where the sensing part 13 is provided. Therefore, the physical quantity measurement apparatus 1 is configured so that a high temperature body does not come close to the sensing part 13 during soldering, nor does heat transfer easily from a high temperature body to the sensing part 13. This avoids changes in the characteristics of the sensing part 13, failure, and a reduction in reliability. In addition, the fact that the surface electrode 17 is provided only on the back surface 10b avoids flux (a soldering accelerator) from adhering to the sensing part 13 or the like during soldering with the second substrate 20. The fact that the surface electrode 17 is provided only on the back surface 10b also enables automation of the soldering with the second substrate 20 by a machine. Here, to solder with the side electrode 27 provided on the side of the second substrate 20, the surface electrode 17 is provided so as to overlap with the side of the second substrate 20 when assembled as the physical quantity measurement apparatus 1. In other words, when the first substrate 10 is viewed from the z-axis direction, an overlapping area exists between the first substrate 10 and the second substrate 20, such as the area A1 or area A2 in FIG. 3, and the surface electrode 17 is arranged so that a portion thereof is included in the overlapping area. The first substrate 10 preferably has sufficient thickness (length in the z-axis direction) to enhance the effect of not transferring heat.

Furthermore, the fact that the surface electrode 17 is provided only on the back surface 10b enables provision of numerous components on the front surface 10a of the first substrate 10. The sensing part 13 and other components configuring the gas sensor can be placed anywhere on the front surface 10a, including the above-described overlapping area. Therefore, at least a portion of the sensing part 13 and the components may be provided to be in overlap with the side electrode 27 when the first substrate 10 is viewed from a direction perpendicular to the front surface 10a or the back surface 10b of the first substrate 10. In the example in FIG. 3, the light emitting element 11 is provided on the front surface 10a so as to overlap the edge of the area A1. When the first substrate 10 is viewed from the z-axis direction, the edge of the area A1 corresponds to the connecting portion between the surface electrode 17 and the side electrode 27. The light emitting element 11 is therefore provided so as to overlap the side electrode 27. Even with this arrangement, during soldering the light emitting element 11 is not affected by heat, nor does flux adhere, as described above. The physical quantity measurement apparatus 1 according to the present embodiment can thus increase the degree of freedom in the arrangement of the sensing part 13 and components on the first substrate 10. The area of the front surface 10a of the first substrate 10 on which the sensing part 13 and components are arranged can therefore be reduced, enabling a reduction in size in the physical quantity measurement apparatus 1. Here, for example, if the components are heat-resistant, they can be arranged on the back surface 10b instead of the front surface 10a. In the case in which some of the components of the gas sensor are arranged on the back surface 10b, the physical quantity measurement apparatus 1 can be further reduced in size.

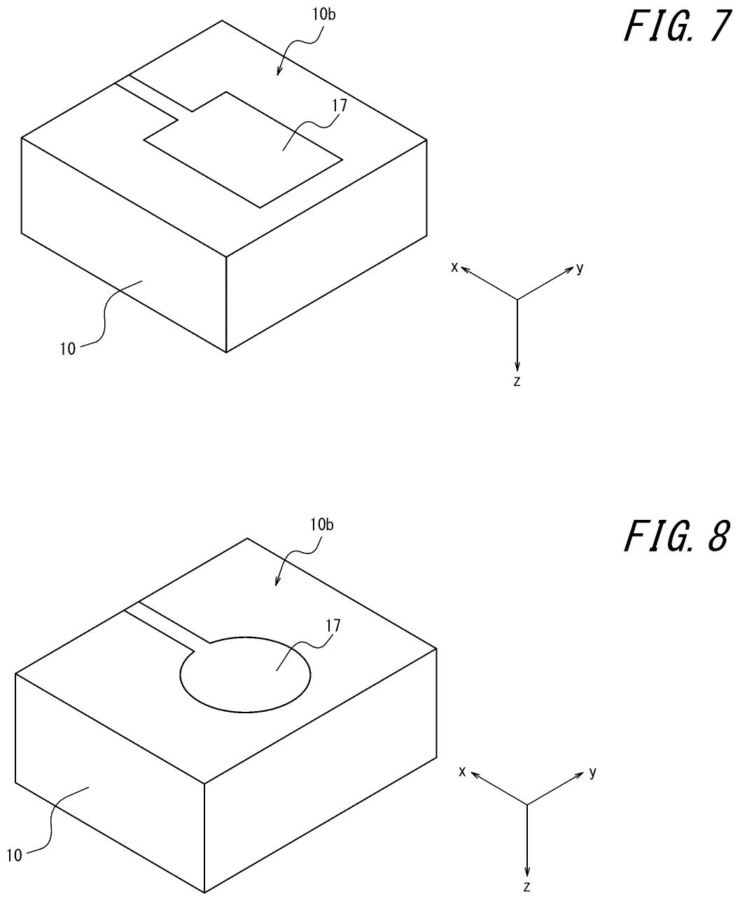

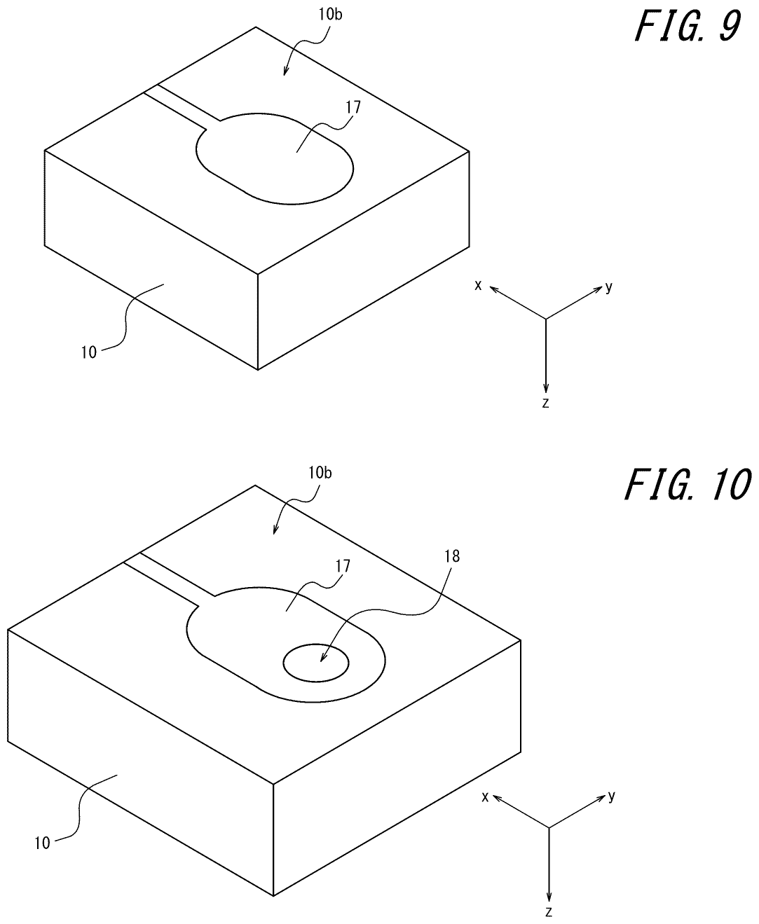

The shape of the surface electrode 17 is not limited. The surface electrode 17 may be rectangular, as illustrated in FIG. 7, for example, or polygonal. The surface electrode 17 may, for example, be a perfect circle as illustrated in FIG. 8, an oval as illustrated in FIG. 9, or an ellipse. The surface electrode 17 may also include a through-hole electrode 18, for example, as illustrated in FIG. 10. The inclusion of the through-hole electrode 18 can strengthen the bonding between the surface electrode 17 and the first substrate 10. By applying a test probe or the like to the through-hole electrode 18 from the front surface 10a side of the first substrate 10 after connecting the first substrate 10 to the second substrate 20 by soldering, an electrical connection between the surface electrode 17, including the through-hole electrode 18, and the test probe can be obtained. The surface electrode 17 may be manufactured by known methods, such as by forming a resist pattern and subsequently plating.

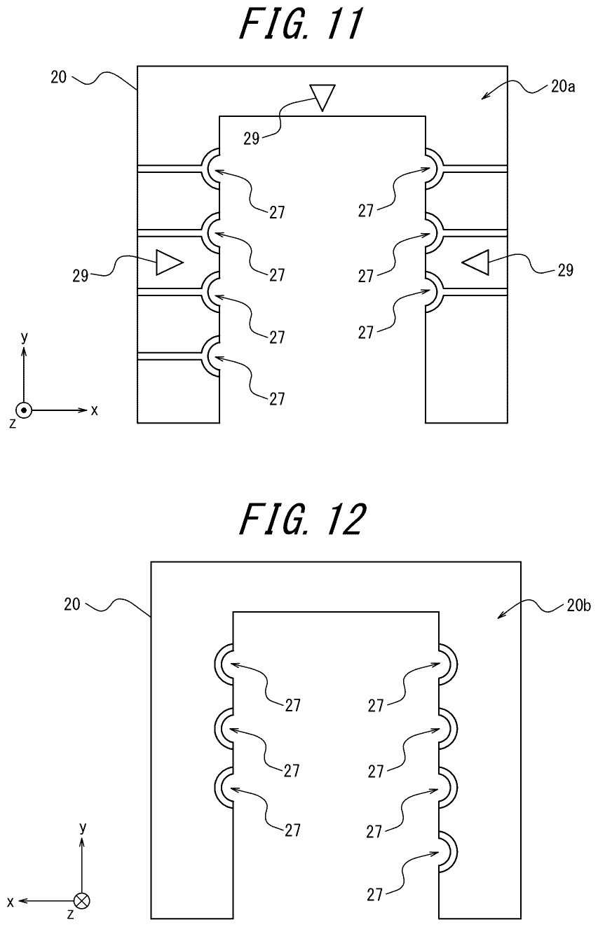

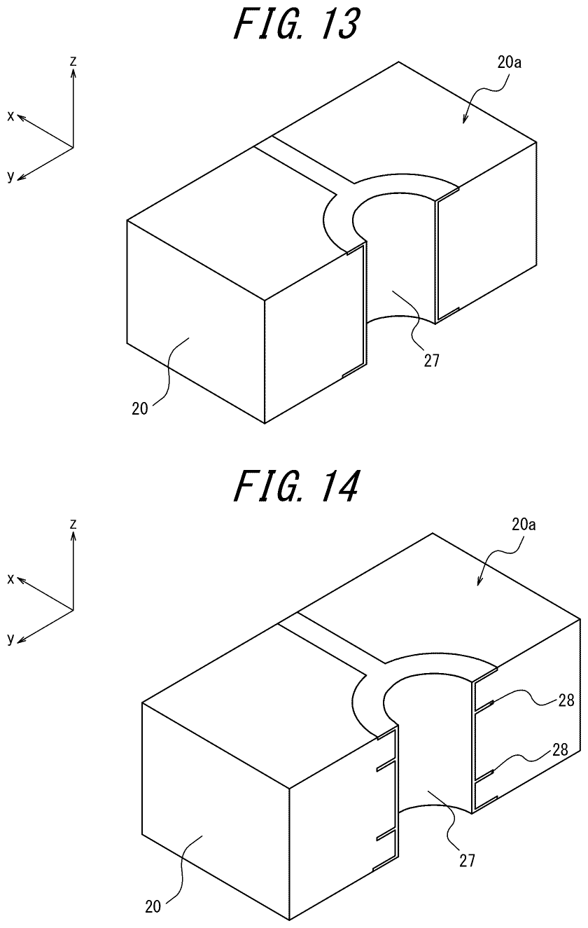

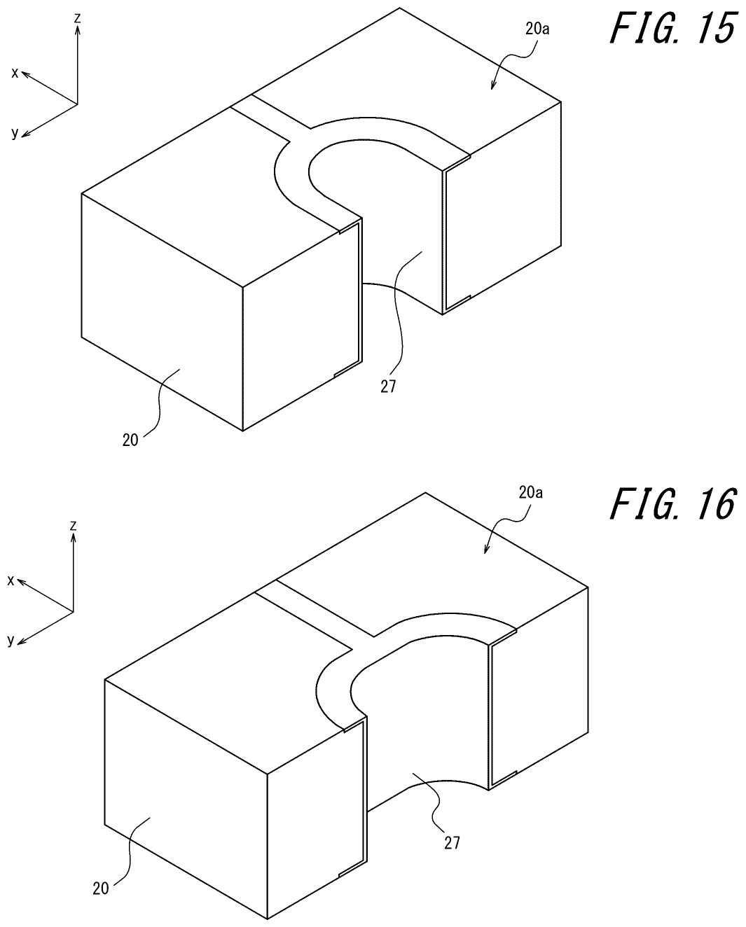

FIG. 11 is a plan view illustrating the front surface 20a of the second substrate 20. FIG. 12 is a plan view illustrating the back surface 20b of the second substrate 20. As described above, the second substrate 20 has side electrodes 27 on a portion of the side between the front surface 20a and the back surface 20b. The shape of the side electrode 27 is not limited to a particular shape but is a castellated hole in the present embodiment. In other words, in the present embodiment, a side electrode 27 having the shape of a hole that is half-open as viewed from the z-axis direction is used, as illustrated in FIGS. 11 and 12. Here, the shape of the hole in the castellated holes is not particularly limited. The shape of the hole may be a half circle, for example, as illustrated in FIG. 13. Annular rings 28 may be provided in a metal foil layer inside the substrate, for example, as illustrated in FIG. 14, in a case in which the second substrate 20 is configured to have multiple layers. This configuration can strengthen the bonding between the castellated hole and the second substrate 20. The shape of the hole may be a portion of an oval elongated in the direction of the recess, for example, as illustrated in FIG. 15. The shape of the hole may be a portion of an oval elongated in the cross-sectional direction, for example, as illustrated in FIG. 16. The side electrode 27 may be manufactured by known methods, such as by forming a resist pattern and subsequently plating.

At least one of the first substrate 10 and the second substrate 20 may have at least one of a guide and mark used to align the first substrate 10 and the second substrate 20. In the present embodiment, the first substrate 10 has three marks 19 on the front surface 10a, as illustrated in FIG. 4, and the second substrate 20 has three marks 29 on the front surface 20a, as illustrated in FIG. 11. By aligning the marks 19 on the first substrate 10 with the marks 29 on the second substrate 20, soldering can be performed in the correct position, and the physical quantity measurement apparatus 1 can easily be assembled. The marks 19 and marks 29 are, for example, formed by printing. Here, guides may be used instead of the marks 19 and marks 29. The guides are convexities or concavities, for example, that enable alignment by physical movement restriction, fitting, or the like.

As described above, the aforementioned configuration of the physical quantity measurement apparatus 1 according to the present embodiment enables measurement with high accuracy by avoiding thermal damage to the sensing part 13.

Although embodiments of the present disclosure have been described through drawings and examples, it is to be noted that various changes and modifications will be apparent to those skilled in the art based on the present disclosure. Therefore, such changes and modifications are to be understood as included within the scope of the present disclosure. For example, the functions and the like included in the various components may be reordered in any logically consistent way. Furthermore, components may be combined into one or divided.

In the above embodiments, it was explained that the sensing part 13 is part of a gas sensor, but the sensing part 13 may be part of various types of sensors that measure the length, mass, current, temperature, or the like of the object to be measured. In the above embodiments, the sensing part 13 has been described particularly as being part of an NDIR gas sensor in which the detection element 12 is a photodetector, but other types of gas sensors may also be used. For example, a photoacoustic gas sensor measures the gas concentration by using a high-performance microphone to pick up, as sound, the vibrations of gas molecules that have absorbed infrared light. In a case in which the sensing part 13 is part of a photoacoustic gas sensor, the detection element 12 is a microphone, for example.

### REFERENCE SIGNS LIST

1 Physical quantity measurement apparatus
10 First substrate
10a Front surface
10b Back surface
11 Light emitting element
12 Detection element
13 Sensing part
15 Light guiding member
17 Surface electrode
18 Through-hole electrode
19 Mark
20 Second substrate
20a Front surface
20b Back surface
27 Side electrode
28 Annular ring
29 Mark
30 IC

1. A physical quantity measurement apparatus that comprises a first substrate and a second substrate and measures a physical quantity of an object to be measured, wherein
the first substrate includes a sensing part on a front surface and a surface electrode on a back surface, the sensing part being configured to output a signal according to the physical quantity,
the second substrate includes a side electrode,
when the first substrate is viewed from a direction perpendicular to the front surface or the back surface of the first substrate, at least a portion of the first substrate overlaps the second substrate, and
the surface electrode is electrically connected to the side electrode.

2. The physical quantity measurement apparatus according to claim 1, wherein the side electrode is a castellated hole.

3. The physical quantity measurement apparatus according to claim 1, wherein the surface electrode includes a through-hole electrode.

4. The physical quantity measurement apparatus according to claim 1, wherein the sensing part includes a light emitting element and a detection element that detects an amount of absorption, by the object to be measured, of light emitted from the light emitting element, and the physical quantity is measured on the basis of the amount of absorption.

5. The physical quantity measurement apparatus according to claim 4, wherein the light is infrared light.

6. The physical quantity measurement apparatus according to claim 1, wherein the physical quantity is a concentration of a gas to be detected.

7. The physical quantity measurement apparatus according to claim 1, wherein at least one of the first substrate and the second substrate includes at least one of a guide and a mark used to align the first substrate and the second substrate.

8. The physical quantity measurement apparatus according to claim 1, wherein
the first substrate includes the sensing part and a component on the front surface, and
at least a portion of the sensing part and the component is provided to be in overlap with the side electrode when the first substrate is viewed from a direction perpendicular to the front surface or the back surface of the first substrate.
